(19) United States
(12) Patent Application Publication
(10) Pub. No.: US 2012/0183519 A1
(43) Pub. Date: Jul. 19, 2012

(54) TREATMENT OF ERECTILE DYSFUNCTION USING PLATELET-RICH PLASMA

(75) Inventor: Matthew SWIFT, Fort Wayne, IN (US)

(73) Assignee: Biomet Biologics, LLC, Warsaw, IN (US)

(21) Appl. No.: 13/006,149

(22) Filed: Jan. 13, 2011

(51) Int. Cl. A61K 35/16; A61K 38/20; A61P 15/00; A61K 38/19

(52) U.S. Cl. 424/93.72; 424/85.1; 424/85.2

(57) ABSTRACT

Methods, apparatus, and compositions related to a method of treating erectile dysfunction in a subject, the method comprising administering a composition comprising platelet-rich plasma at or proximate to ischemic tissue comprising the corpora cavernosa or corpus spongiosum.

### INTRODUCTION

The present technology relates to methods for treating erectile dysfunction (ED), including systems, apparatus, and compositions relating to such methods.

Intercourse in mammals requires physiological stimulation of male erectile tissues in the penis to mechanically support transfer of sperm from male to female. Defects preventing an appropriate erectile tissue response can interfere with reproductive capability. In humans, erectile dysfunction is considered a disease state referred to as the condition of “impotence.” This condition impacts the quality of life for patients and their partners.

Erection of the penis is a hemodynamic phenomenon involving tissue of the corpora cavernosa and the corpus spongiosum. This tissue is a complex admixture of smooth muscle, endothelial cells, fibroblasts, and nerves that interact under stimulatory conditions in order to enhance and maintain an accessory blood supply imparting rigidity. Given the need for control of blood flow during this response, it is evident that vascular insufficiency can suppress erectile capability. In fact, vascular insufficiency may be a common pathomechanism of erectile dysfunction. It is estimated that from 50% to 75% of the instances of erectile dysfunction can be attributed at least in part to a reduced blood flow to the penis, typically due to an underlying vascular disease.

Pharmacological methods have been developed to treat erectile dysfunction including the use of vasodilators. Orally administered vasodilators are among the most widely accepted treatments. Such vasodilators include sildenafil (Viagra™). However, vasodilators induce temporary erections following administration and do not address or treat the basic vascular/cavernosal pathology causing erectile dysfunction.

Accordingly, treatments that address tissue defects and/or vascular disease aspects of erectile dysfunction may provide a longer term therapy or even provide a permanent effect in comparison to other treatments.

### SUMMARY

The present technology includes methods, apparatus, and compositions that relate to treating erectile dysfunction in a subject using platelet-rich plasma, where the platelet-rich plasma can be derived from the subject, for example. Methods of treating erectile dysfunction in a subject include administering a composition comprising platelet-rich plasma at or proximate to ischemic tissue comprising the corpora cavernosa or corpus spongiosum. The platelet-rich plasma promotes angiogenesis to form new blood vessels and/or extend preexisting blood vessels into the ischemic tissue. The platelet-rich plasma can be isolated by density fractionation of a blood material, such as whole blood, a blood fraction, or bone marrow aspirate.

In some embodiments, isolating platelet-rich plasma by density fractionation of the blood material includes loading the blood material into a tube comprising a buoy disposed in the tube. The buoy has a density such that the buoy is operable to reach an equilibrium position upon centrifugation of the blood component in the tube. The equilibrium position is between a platelet-rich plasma fraction and a second fraction. The platelet-rich plasma fraction has a concentration of platelets greater than the concentration of platelets in the second fraction. The tube is then centrifuged to define the platelet-rich plasma fraction and the platelet-rich plasma fraction is removed from the tube.

In some embodiments, administering a composition comprising platelet-rich plasma at or proximate to ischemic tissue comprising the corpora cavernosa or corpus spongiosum can include injecting serial aliquots of the platelet-rich plasma along ischemic regions of the corpora cavernosa or corpus spongiosum. The present methods can further include administering a platelet activator such as thrombin, calcium, collagen, epinephrine, adenosine diphosphate, and mixtures thereof. And in some cases, the administering can include fibrinogen. An isolated angiogenic factor can also be administered at or proximate to the ischemic tissue comprising the corpora cavernosa or corpus spongiosum.

### BRIEF DESCRIPTION OF THE DRAWINGS

FIG. 1 is a partial cross-sectional view of a representative device for preparing platelet-rich plasma according to methods of the present technology.

FIG. 2 is a diagrammatic illustration of a method of treating erectile dysfunction according to the present technology.

FIG. 3 is a diagrammatic illustration of another method of treating erectile dysfunction according to the present technology.

FIG. 4 is a diagrammatic illustration of another method of treating erectile dysfunction according to the present technology.

It should be noted that the figures set forth herein are intended to exemplify the general characteristics of materials and methods among those of the present technology, for the purpose of the description of certain embodiments. These figures may not precisely reflect the characteristics of any given embodiment, and are not necessarily intended to define or limit specific embodiments within the scope of this technology.

### DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Description.

The present technology relates to methods of treating erectile dysfunction. Methods include administering a composition comprising platelet-rich plasma at or proximate to ischemic tissue comprising the corpora cavernosa or corpus spongiosum. Platelet-rich plasma consequently promotes angiogenesis to provide new blood vessels and/or the extension of preexisting blood vessels to the ischemic tissue. In this manner, tissue defects or vascular disease aspects effecting erectile dysfunction can be treated to provide a long-term therapy or even a permanent effect. Improved vascularization of the corpora cavernosa or corpus spongiosum can alleviate symptoms of erectile dysfunction and can enhance aspects of other erectile dysfunction treatments, such as administration of vasodilators.

In various embodiments, without limiting the scope or use of the present technology, it is believed that the present methods treat erectile dysfunction caused at least in part by lack of tissue vascularity. For example, platelet-rich plasma derived from autologous whole blood or bone marrow aspirate can be injected into the ischemic tissue of the penis to induce angiogenesis at one or more sites in order to improve blood flow.

Angiogenesis is generally referred to as the growth of new blood vessels and/or the extension of preexisting blood vessels and is an important natural process in the body. For example, angiogenesis occurs following an injury as part of normal wound healing in order to restore blood flow to damaged tissues. In various embodiments, without limiting the scope or use of the present technology, it is believed that the present methods and compositions can promote and enhance angiogenesis in damaged or avascular tissue. In this way, tumescence can be partially or fully restored to the affected site to treat erectile dysfunction.

Platelet-rich plasma, sometimes abbreviated as “PRP” in the literature, refers to a blood fraction having a concentration of platelets greater than the concentration of platelets typically found in whole blood. For example, whole blood, such as peripheral blood, typically includes platelet concentrations of about 150,000 to about 450,000 per μL of blood (i.e., about 150-450×10⁹/L), whereas platelet-rich plasma includes platelet concentrations ranging from about 500,000 to about 1,200,000 per μL or more (i.e., ≧500-1200×10⁹/L).

Platelet-rich plasma can be prepared by concentrating platelets from a blood material (e.g., whole blood, blood fractions, and bone marrow aspirate) obtained using autologous, allogenic, or pooled sources of platelets and/or plasma. Platelet-rich plasma may be formed from a variety of sources, including various human and animal sources. For example, platelet-rich plasma can be prepared from a subject's own blood or bone marrow in order to provide an autologous product, thereby minimizing the potential for contamination and immunogenic issues.

Platelet-rich plasma can be prepared from whole blood. Whole blood contains various proteins, cells including red blood cells, white blood cells, and platelets, and other components. For example, whole blood can be separated into blood fractions, such as platelet-rich plasma and platelet-poor plasma, using a range of methods. Compositions comprising platelet-rich plasma can include (unactivated) platelets, activated platelets, platelet releasate, and combinations thereof.

Platelets, also called thrombocytes, are derived from fragmentation of precursor bone marrow megakaryocytes, including cytoplasmic portions thereof They have no nucleus for replication and their lifespan is about five to nine days. Platelets are involved in the hemostatic process and release several initiators in the clotting cascade. Platelets contain a store of cytokines in a-granules and release these cytokines during wound healing.

In response to platelet aggregation or following contact with connective tissue, as may occur following injury or surgery, the cell membrane of a platelet is activated to secrete the contents of the α-granules. A wide variety of cytokines are released by activated platelets. These cytokines can include platelet factor 4, transforming growth factor-β1, platelet-derived growth factor, fibronectin, B-thromboglobulin, von Willebrand factor, fibrinogen, and coagulation factors V and XIII. These cytokines serve a number of different functions in the healing process, including helping to stimulate cell division. They also work as chemotactic factors for cells including mesenchymal cells, monocytes, and fibroblasts among others. The internal contents of the platelet, including cytokines, can be referred to as the platelet releasate, which has the potential to affect the function of other cells.

A composition comprising platelet-rich plasma may be prepared using various methods of isolating or concentrating platelets from whole blood or platelet-containing blood fractions. Platelet-rich plasma may also be isolated using any of a variety of devices described, including those in U.S. Pat. No. 6,398,972, Blasetti et al., issued Jun. 4, 2002; U.S. Pat. No. 6,649,072, Brandt et al., issued Nov. 18, 2003; U.S. Pat. No. 6,790,371, Dolocek, issued Sep. 14, 2004; U.S. Pat. No. 7,011,852, Sukavaneshvar et al., issued Mar. 14, 2006; U.S. Pat. No. 7,179,391, Leach et al., issued Feb. 20, 2007 (incorporated by reference herein); U.S. Pat. No. 7,374,678, Leach et al., issued May 20, 2008 (incorporated by reference herein); U.S. Pat. No. 7,223,346, Dorian et al., issued May 29, 2007 (incorporated by reference herein); and U.S. Pat. No. 7,708,152, Dorian et al., issued May 4, 2010 (incorporated by reference herein).

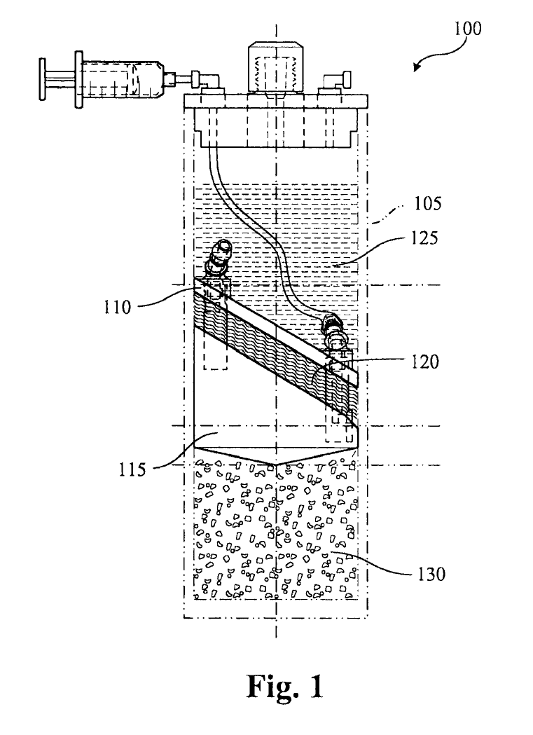

Referring now to FIG. 1, one example of a device that may be used for isolating platelet-rich plasma is shown. In this regard, the device 100 includes a container 105, such as a tube, that is placed in a centrifuge after being filled with a blood material (e.g., whole blood or a blood component). The container 105 includes a buoy system having an isolator 110 and a buoy 115. The buoy 115 has a selected density which is tuned to reach a selected equilibrium position upon centrifugation; this position lies between a more dense blood fraction and a less dense blood fraction. During centrifugation, the buoy 115 separates the whole blood or a blood component within the container 105 into at least two fractions, without substantially commingling the fractions, by sedimenting to a position between the two fractions. In this regard, the isolator 110 and the buoy 115 define a layer comprising platelet-rich plasma 120, while less dense platelet-poor plasma 125 generally fractionates above the isolator 110, and more dense red blood cells 130 generally fractionate below the buoy 115. Following centrifugation of the device 100, a syringe or tube may be interconnected with a portion of the buoy system to extract the platelet-rich plasma. Commercially available embodiments of such devices include the GPS™ II Platelet Concentrate System, from Biomet Biologics, LLC (Warsaw, Ind., USA) and GPS™ III Platelet Separation System, from Biomet Biologics, LLC (Warsaw, Ind., USA).

In addition to the GPS™ Platelet Concentrate and Separation Systems, a variety of other commercially available devices may be used to isolate platelet-rich plasma at step 220, including the Magellan™ Autologous Platelet Separator System, commercially available from Medtronic, Inc. (Minneapolis, Minn., USA); SmartPReP™, commercially available from Harvest Technologies Corporation (Plymouth, Mass., USA); DePuy (Warsaw, Ind., USA); the AutoloGel™ Process, commercially available from Cytomedix, Inc. (Rockville, Md., USA); the GenesisCS System, commercially available from EmCyte Corporation (Fort Myers, Fla., USA); and the PCCS System, commercially available from Biomet 3i, Inc. (Palm Beach Gardens, Fla., USA).

Another example of a device that may be used to isolate platelet-rich plasma includes a centrifugal drum separator and an erythrocyte (i.e., red blood cell) capture trap. In one embodiment, the walls of the centrifugal drum separator are coated with a depth filter having pores and passageways that are sized to receive and entrap erythrocytes. A blood material is placed in the centrifugal drum, and the drum is spun along its axis at sufficient speed so as to force erythrocytes from the blood into the depth filter. After spinning, the erythrocytes remain in the filter and the remaining platelet-rich plasma is extracted. The platelet-rich plasma may be concentrated by desiccation. Embodiments of such a device include the Vortech™ Concentration System (Biomet Biologics, Inc., Warsaw, Ind.), and include devices as disclosed in U.S. Application Publication No. 2006/0175244, Dorian et al., published Aug. 10, 2006 and U.S. Pat. No. 7,708,152, Dorian et al., issued May 4, 2010, which are hereby incorporated by reference. Such devices may be used to prepare platelet-rich plasma in lieu of or in addition to the various methods described herein.

Other methods may be used to isolate platelet-rich plasma. For example, a blood material can be centrifuged without using a buoy system, whole blood can be centrifuged in multiple stages, continuous-flow centrifugation can be used, and filtration can be used. In addition, platelet-rich plasma can be produced by separating plasma from red blood cells using a slow speed centrifugation step to prevent pelleting of the platelets. In some embodiments, a buffy coat fraction formed from centrifuged blood can be separated from remaining plasma and resuspended to form platelet-rich plasma including white blood cells. Affinity column purification of platelets can also be used. Other devices for isolating platelet-rich plasma use high speed centrifugation to pellet the platelets and red blood cells. The pelleted platelets are then resuspended using some of the plasma supernatant or another suitable solution.

The concentration of platelets within the platelet-rich plasma may vary depending on the concentration of platelets in the source whole blood or blood component as well as the particular device and method employed in preparation of the platelet-rich plasma. For example, in some embodiments, the platelet concentration in the platelet-rich plasma can be from about 3-fold to about 10-fold greater than the platelet concentration in the source whole blood or blood component. In various embodiments, devices may be used to generate platelet-rich plasma that includes a platelet concentration up to about 8-fold higher than whole blood. Furthermore, preparations of platelet-rich plasma can contain cytokines, growth factors, and other proteins and molecules in addition to those contained within or fractionating with the platelets. For example, pelleted platelets that are resuspended in whole or in part with a plasma supernatant can contain cytokines and growth factors from the plasma supernatant.

The blood material used to prepare platelet-rich plasma can be mixed with an anticoagulant prior to preparation of the platelet-rich plasma or at one or more points during preparation. Suitable anticoagulants include those known in the art, such as heparin, citrate phosphate dextrose (CPD), ethylenediaminetetraacetic acid (EDTA), acid citrate dextrose solution (ACD), and mixtures thereof. The anticoagulant typically includes a chelating agent (e.g., citrate, EDTA) to complex free calcium ions. For example, the anticoagulant may be placed in a syringe used for drawing blood from the subject, or may be mixed with the blood after it is drawn.

In some embodiments, the anticoagulant comprises acid-citrate-dextrose (ACD), which is a solution of citric acid, sodium citrate, and dextrose in water, that can be used to preserve whole blood and/or the blood component. For example, the anticoagulant may include ACD-A, which includes per 1000 mL: total citrate (as citric acid, anhydrous (C₆H₈O₇)) about 20.59 g to 22.75 g, dextrose (C₆H₁₂0₆*H₂O) about 23.28 g to 25.73 g, and sodium (Na) about 4.90 g to 5.42 g. In some embodiments, ACD-B is used, which includes per 1000 mL: total citrate (as citric acid, anhydrous (C₆H₈O₇)) about 12.37 g to 13.67 g, dextrose (C₆H₁₂O₆*H₂O) about 13.96 g to 15.44 g, and sodium (Na) about 2.94 g to 3.25 g. In some embodiments, whole blood can be anticoagulated with about a 1/10^{th} volume to about a 1/7^{th} volume of ACD-A.

Optional materials, such as bone marrow aspirate, platelet activators, and angiogenic factors may be co-administered with the platelet-rich plasma. With respect to the use of bone marrow aspirate, this material can be harvested by needle aspiration of bone marrow and can be harvested from the posterior illiac crest, anterior illiac crest, sternum, or tibia. Preferably, the bone marrow aspirate is autologous or allogenic. Bone marrow aspirate can be administered just prior to the administration of the platelet-rich plasma, concomitant with administration the platelet-rich plasma, or following administration of the platelet-rich plasma to the subject. In some embodiments, bone marrow aspirate can be used in preparation of platelet-rich plasma and can be combined with whole blood and/or a blood component that is then used in preparation of platelet-rich plasma.

One or more platelet activators can be included in the composition comprising platelet-rich plasma or the platelet activator(s) can be separately administered. The platelet activators serve to release cytokines and growth factors within the platelets; e.g., forming a platelet releasate. Platelet activators include thrombin, calcium, collagen, epinephrine, adenosine diphosphate, and mixtures thereof Activation of the platelets in platelet-rich plasma using a platelet activator can be performed just prior to administration of the platelet-rich plasma to the subject, concomitant with administration the platelet-rich plasma to the subject, or following administration of the platelet-rich plasma to the subject.

Platelet-rich plasma can form a fibrin tissue adhesive or gel following activation; e.g., by thrombin and calcium. Activation results in release of the various cytokines by the platelets and also starts a clotting reaction among various components of the plasma fraction. The clotting reaction can result in the formation of a platelet gel. In some embodiments, the composition comprising platelet-rich plasma used in the present methods can include a platelet releasate formed following contact with platelet activator, in addition to platelets themselves. The releasate comprises the various cytokines released by degranulating platelets following activation.

Thrombin, a multifunctional serine protease, can be generated from prothrombin by enzymatic cleavage of two sites on prothrombin by activated Factor X (Xa). Factor Xa activity is enhanced by binding to activated Factor V (Va), termed the prothrombinase complex. Once formed, thrombin-mediated proteolytic digestion of fibrinogen into fibrin monomer starts a reaction cascade that can lead to clot formation, which is typically the first step in wound healing. Thrombin can also function as a chemo-attractant to cells involved in wound healing and the resulting fibrin network has several functions including acting as a scaffold for collagen-producing fibroblasts, increasing phagocytosis, promoting angiogenesis, and binding growth factors that can further support the healing process. Platelets are also activated from the nonbinding to the binding mode. For example, as a procoagulant, thrombin plays an important role in the arrest of bleeding; i.e., physiological hemostasis.

In some embodiments, thrombin used in the present methods can be prepared using a device as disclosed by U.S. Pat. No. 7,694,828, Swift et al., issued Apr. 13, 2010. An example of such a device is the Clotalyst™ Autologous Clotting Factor System, sold by Biomet Biologics LLC, Warsaw, Ind., USA.

In some embodiments, the composition comprising platelet-rich plasma does not include any platelet activators or include platelet releasate. Collagen, a component of connective tissues, is an activator of platelets. Accordingly, when the platelet-rich plasma composition is introduced at or proximate to connective tissue, platelets in the composition may bind to the native collagen and be activated. In this way, there is no need for administering an exogenous activator such as thrombin, for example.

Administering the composition comprising platelet-rich plasma can further include administering various angiogenic factors. These angiogenic factors may function in concert with the platelet-rich plasma in promoting the growth of new blood vessels and/or the extension of preexisting blood vessels. Examples of angiogenic factors include: angiogenin, angiopoietin-1, del-1 protein, fibroblast growth factors such as acidic FGF (also known as aFGF or FGF-1) and basic FGF (also known as bFGF or FGF-2), follistatin, granulocyte colony-stimulating factor (G-CSF), hepatocyte growth factor (HGF), interleukin-8 (IL-8), leptin, midkine, placental growth factor, platelet-derived endothelial growth factor (PD-ECGF), platelet-derived growth factor (PDGF), pleiotrophin (PTN), progranulin, proliferin, transforming growth factor alpha (TGF-α), transforming growth factor beta (TGF-β), tumor necrosis factor alpha (TNF-α), vascular endothelial growth factor (VEGF), and vascular permeability factor (VPF). In various embodiments, isolated, recombinant, and/or synthetic angiogenic factors may be used. Angiogenic factors can be administered to a site of ischemic tissue just prior to the administration of the platelet-rich plasma, concomitant with administration the platelet-rich plasma, or following administration of the platelet-rich plasma to the subject.

The composition comprising platelet-rich plasma can be administered at or proximate to ischemic tissue comprising the corpora cavernosa or corpus spongiosum to treat erectile dysfunction in a subject. In this regard, the composition comprising platelet-rich plasma can be injected into the ischemic tissue by means of a syringe, such as injection of serial aliquots along the ischemic regions of the penis to stimulate an angiogenic response in situ. Alternatively, the composition can be administered via a surgical procedure, for example using a catheter. The surgical procedure may include an endoscopic surgical procedure. It should be understood, however, that the step of administering the composition of platelet-rich plasma may employ any biomedically acceptable process or procedure by which the platelet-rich plasma composition is implanted, injected, or otherwise administered in, on, or in proximity to the corpora cavernosa or corpus spongiosum in a subject so as to have a beneficial effect, such as increasing vascularization by promoting angiogenesis. Increase in blood flow in the area may be one measure of a successful treatment, where the increased blood can lessen the symptoms of erectile dysfunction. Successive administration of platelet-rich plasma may be used in some cases to more thoroughly vascularize or revascularize ischemic tissue.

Methods of administering the composition including platelet-rich plasma can further comprise administering thrombin, fibrinogen, and calcium to the subject. The fibrinogen or calcium may be combined with the thrombin or these components may be applied separately. In some cases, applying the tissue sealant comprises co-administering a first solution comprising fibrinogen and the platelet-rich plasma composition, and a second solution comprising thrombin and calcium. In such embodiments, the first solution and second solution are kept separate until administered so that the thrombin does not activate the fibrinogen to form a fibrin matrix until after the solutions are mixed and applied at the treatment site. The solutions may be mixed just before application to the treatment site or may be mixed at the treatment site. For example, a dual barreled syringe may be used to simultaneously administer the two solutions. The thrombin, fibrinogen, and calcium can also be combined together to initiate the clotting cascade at a time prior to application to the site on the subject. As the clotting cascade progresses, a gel-like material starts to form, which can then be applied to the site for use as a tissue sealant with the platelet-rich plasma composition. Where the blood component includes a chelating agent, such as citrate, the composition comprising platelet-rich plasma can include excess calcium to overwhelm the chelating agent and provide free calcium ions for the blood-clotting cascade.

The methods and compositions of the present technology can be used to prepare and administer autologous, homologous, or heterologous materials. For example, bovine thrombin can be used in a method where a composition comprising human platelet-rich plasma is administered to a human. Such materials may also be obtained from a homologous source, such as a compatible human donor. However, it is often desirable to use only autologous materials and autologous clotting factors (e.g., autologous fibrinogen, thrombin, and blood components) in the present methods to reduce the possibility of infection, immune reaction, or other side effects from using a non-autologous source. The present methods can therefore provide treatment methods using solely autologous materials derived from the subject being treated for erectile dysfunction.

The present methods of treatment may also be combined with other treatments for erectile dysfunction, including alprostadil (Caverject), sildenafil (Viagra®), tadalafil (Cialis®), and vardenafil (Levitra®). For example, vascularization or revascularization of ischemic tissue of the corpora cavernosa or corpus spongiosum may improve the ability of the subject to develop or maintain an erection. However, in some cases, the improvement can be further augmented by oral administration of a vasodilator. The requisite dosing of the vasodilator may consequently be reduced affording a corresponding reduction in side effects.

The following specific examples are provided for illustrative purposes of how to make and use the compositions and methods of this technology and, unless explicitly stated otherwise, are not intended to be a representation that given embodiments of this technology have, or have not, been made or tested.

### EXAMPLE 1

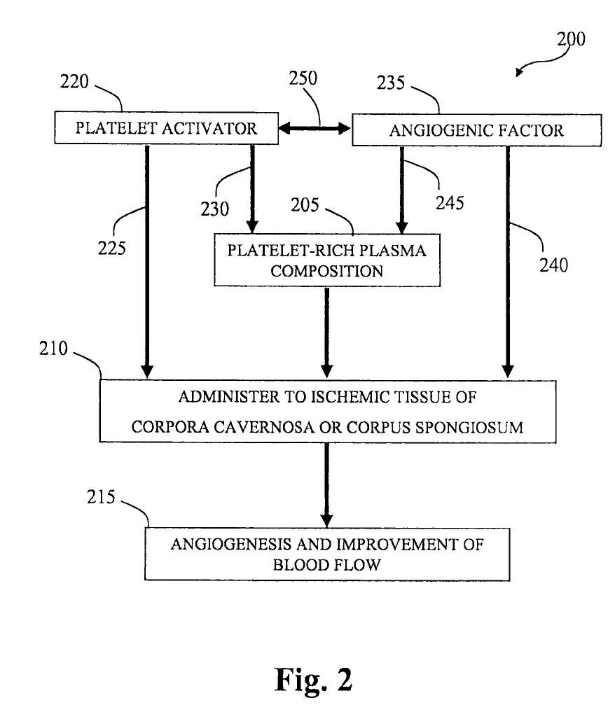

With reference to FIG. 2, a diagrammatic illustration of a method of treating erectile dysfunction in a subject is shown at 200. A composition comprising platelet-rich plasma 205 is administered at or proximate to ischemic tissue comprising the corpora cavernosa or corpus spongiosum of the subject, as shown at step 210. Treatment of the ischemic tissue with the platelet-rich plasma composition subsequently promotes angiogenesis thereby improving blood flow, as shown at 215. Improved blood flow by formation of new vessels and/or extension of preexisting blood vessels partially or fully restores the capacity of the tissue for tumescence and maintenance of an erection.

Optionally, a platelet activator 220 (e.g., thrombin) is co-administered with the platelet-rich plasma 205, as shown by the arrow at 225. Or, the platelet activator 220 can be included in the composition comprising platelet-rich plasma 205, as shown by the arrow at 230. In a similar fashion, an angiogenic factor 235 (e.g., angiogenin) can be co-administered with the platelet-rich plasma 205 (arrow at 240) or included in the composition comprising platelet-rich plasma 205 (arrow at 245). As indicated by 250, a platelet activator 220 and an angiogenic factor 235 can be combined into a single composition for administration to the ischemic tissue at 210 or combined for addition to the platelet-rich plasma composition 205.

### EXAMPLE 2

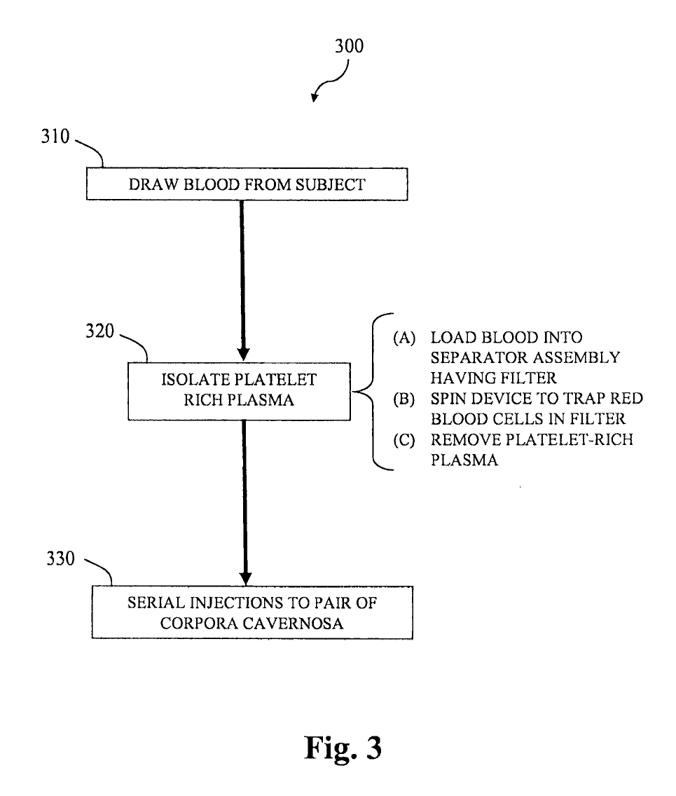

With reference to FIG. 3, a diagrammatic illustration of another method of treating erectile dysfunction in a subject is shown at 300. Whole blood is drawn from a subject having erectile dysfunction as shown at step 310. Platelet-rich plasma is isolated from the subject's own blood at step 320 by using the Vortech™ Concentration System (Biomet Biologics, Inc., Warsaw, Ind.) according to the manufacturer's instructions. Briefly, the whole blood is loaded into a centrifugal drum separator with an erythrocyte (i.e., red blood cell) capture trap. The walls of the centrifugal drum separator are coated with a depth filter having pores and passageways that are sized to receive and entrap erythrocytes. The drum is spun along its axis at sufficient speed so as to force erythrocytes from the blood into the depth filter. After spinning, the erythrocytes remain in the filter and the remaining platelet-rich plasma is extracted. Optionally, the platelet-rich plasma may be concentrated by desiccation. The isolated autologous platelet-rich plasma is then administered to the subject by serial injections along each of the pair of corpus cavernosum within the body of the penis. Subsequent angiogenesis generated by the platelet-rich plasma at least partially remedies the vascular insufficiency responsible for erectile dysfunction in the subject.

### EXAMPLE 3

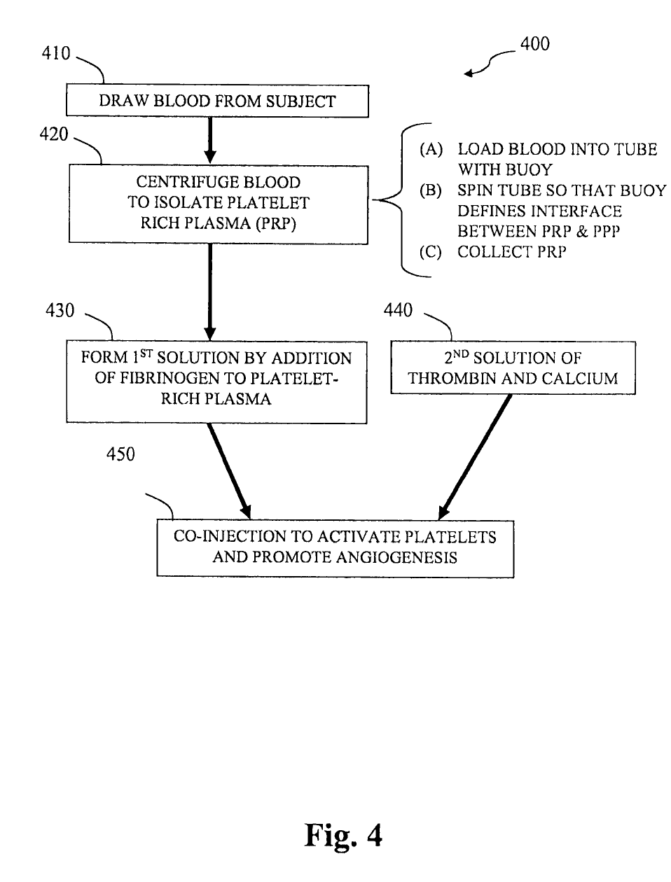

With reference to FIG. 4, a diagrammatic illustration of another method of treating erectile dysfunction in a subject is shown at 400. Blood is drawn from a subject presenting erectile dysfunction due to vascular insufficiency, as shown at 410. The blood is loaded into a tube having a buoy, as shown in FIG. 1. The tube is centrifuged to separate the blood into at least two fractions, without substantially commingling the fractions, by sedimenting to a position between the two fractions. In this regard, the isolator 110 and the buoy 115 define a layer comprising platelet-rich plasma 120, while less dense platelet-poor plasma 125 generally fractionates above the isolator 110, and more dense red blood cells 130 generally fractionate below the buoy 115. The platelet-rich plasma is collected and fibrinogen is added to form a first solution, as shown at 430. The first solution 430 and a second solution 440 of thrombin and calcium are then co-injected into ischemic tissue comprising the corpora cavernosa or corpus spongiosum in the subject. The thrombin and calcium activate the platelets forming a releasate at the injection site which is further immobilized by activation of the fibrin, which forms a fibrin glue. The platelets and platelet releasate are retained at the injection site by the clot, thereby promoting angiogenesis via cytokines and growth factors present therein. Resulting vascularization improves the ability of the subject to maintain an erection.

The examples and other embodiments described herein are exemplary and not intended to be limiting in describing the full scope of compositions and methods of this technology. Equivalent changes, modifications and variations of specific embodiments, materials, compositions and methods may be made within the scope of the present technology, with substantially similar results.

Consideration should be given to the following non-limiting discussion of terminology as used in the present disclosure. The headings (such as “Introduction” and “Summary”) and sub-headings used herein are intended only for general organization of topics within the present disclosure, and are not intended to limit the disclosure of the technology or any aspect thereof In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “desire” or “desirable” refer to embodiments of the technology that afford certain benefits, under certain circumstances. However, other embodiments may also be desirable, under the same or other circumstances. Furthermore, the recitation of one or more desired embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include disclosure of all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

What is claimed is:

1. A method of treating erectile dysfunction in a subject, the method comprising administering a composition comprising platelet-rich plasma at or proximate to ischemic tissue comprising the corpora cavernosa or corpus spongiosum.

2. The method of claim 1, wherein the platelet-rich plasma is derived from the subject.

3. The method of claim 1, wherein the composition comprising platelet-rich plasma is prepared by a process comprising:
obtaining a blood material compatible with the subject; and
isolating platelet-rich plasma by density fractionation of the blood material.

4. The method of claim 3, wherein the blood material comprises whole blood, a blood fraction, or bone marrow aspirate.

5. The method of claim 3, wherein the blood material further comprises an anticoagulant.

6. The method of claim 3, wherein isolating platelet-rich plasma by density fractionation of the blood material comprises:
loading the blood material into a tube comprising a buoy disposed in the tube, wherein the buoy has a density such that the buoy is operable to reach an equilibrium position upon centrifugation of the blood material in the tube, the position being between a platelet-rich plasma fraction and a second fraction, wherein the platelet-rich plasma fraction has a concentration of platelets greater than the concentration of platelets in the second fraction;
centrifuging the tube to define the platelet-rich plasma fraction; and
removing the platelet-rich plasma fraction.

7. The method of claim 6, wherein the blood material comprises whole blood and the tube further comprises an isolator operable to define an interface between a fraction that comprises platelet-rich plasma and platelet-poor plasma.

8. The method of claim 3, wherein isolating platelet-rich plasma by density fractionation of the blood component comprises:
loading the blood material into a tube comprising two buoys, wherein the buoys have densities such that the buoys are operable to reach equilibrium positions upon centrifugation of the blood material in the tube, the positions being between three fractions including a platelet-rich plasma, wherein the platelet-rich plasma fraction has a concentration of platelets greater than the other two fractions;
centrifuging the separator to define the platelet-rich plasma fraction; and
removing the platelet-rich plasma fraction.

9. The method of claim 8, wherein centrifuging the separator to define the platelet-rich plasma fraction further defines a platelet-poor plasma fraction and a red blood cell fraction.

10. The method of claim 3, wherein obtaining a blood material compatible with the subject comprises:
identifying and obtaining donor blood compatible with the subject by matching blood cell surface antigens; or
obtaining the blood component from the subject.

11. The method of claim 1, wherein the composition comprising platelet-rich plasma is prepared by a process comprising:
obtaining from the subject a tissue comprising whole blood, bone marrow aspirate, or a mixture of whole blood and bone marrow aspirate;
loading the tissue and an anticoagulant into a tube comprising a buoy disposed in the tube, wherein the buoy has a density such that the buoy reaches an equilibrium position upon centrifugation of the tissue in the tube, the position being between a platelet-rich plasma fraction and a second fraction, wherein the platelet-rich plasma fraction has a concentration of platelets greater than the concentration of platelets in the second fraction;
centrifuging the tube to define the platelet-rich plasma fraction; and
collecting the platelet-rich plasma layer.

12. The method of claim 1, wherein administering a composition comprising platelet-rich plasma at or proximate to ischemic tissue comprising the corpora cavernosa or corpus spongiosum comprises injecting serial aliquots of the platelet-rich plasma along ischemic regions of the corpora cavernosa or corpus spongiosum.

13. The method of claim 1, further comprising administering a platelet activator at or proximate to ischemic tissue comprising the corpora cavernosa or corpus spongiosum, wherein the platelet activator is selected from the group consisting of thrombin, calcium, collagen, epinephrine, adenosine diphosphate, and mixtures thereof.

14. The method of claim 13, wherein the thrombin is autologous to the subject.

15. The method of claim 13, wherein administering a platelet activator further comprises administering fibrinogen.

16. The method of claim 15, wherein administering a composition comprising platelet-rich plasma and administering a platelet activator at or proximate to ischemic tissue comprising the corpora cavernosa or corpus spongiosum comprises co-administering a first solution comprising fibrinogen and the composition comprising platelet-rich plasma, and a second solution comprising thrombin and calcium.

17. The method of claim 1, further comprising administering an isolated angiogenic factor at or proximate to ischemic tissue comprising the corpora cavernosa or corpus spongiosum, wherein the isolated angiogenic factor is selected from the group consisting of angiogenin, angiopoietin-1, del-1 protein, acidic fibroblast growth factor (aFGF or FGF-1), basic fibroblast growth factor (bFGF or FGF-2), follistatin, granulocyte colony-stimulating factor (G-CSF), hepatocyte growth factor (HGF), interleukin-8 (IL-8), leptin, midkine, placental growth factor, platelet-derived endothelial growth factor (PD-ECGF), platelet-derived growth factor (PDGF), pleiotrophin (PTN), progranulin, proliferin, transforming growth factor alpha (TGF-α), transforming growth factor beta (TGF-β), tumor necrosis factor alpha (TNF-α), vascular endothelial growth factor (VEGF), vascular permeability factor (VPF), and combinations thereof.

18. The method of claim 17, wherein the composition comprising platelet-rich plasma comprises the isolated angiogenic factor.
